(19) United States
(12) Patent Application Publication
(10) Pub. No.: US 2026/0248453 A1
(43) Pub. Date: Aug. 27, 2026

(54) REUSABLE APPLICATOR MONITORING SYSTEM

(71) Applicant: Glucovation, Inc., Carlsbad, CA (US)

(72) Inventor: Jeffrey Suri, Carlsbad, CA (US)

(73) Assignee: Glucovation, Inc., Carlsbad, CA (US)

(21) Appl. No.: 19/163,063

(22) PCT Filed: Mar. 7, 2024
(86) PCT No.: PCT/US2024/018862

§ 371 Date: Sep. 8, 2025

Related U.S. Application Data
(60) Provisional application No. 63/489,491, filed on Mar. 10, 2023.

(51) Int. Cl. A61B 5/00; A61B 5/1473

(52) U.S. Cl. CPC A61B 5/6849; A61B 5/1473; A61B 5/6833; A61B 2560/063

(57) ABSTRACT

Disclosed herein are systems, methods and devices related to analyte monitoring devices, such as monitoring devices that effect continuous analyte monitoring. Systems, methods and devices herein often comprise a delivery device and a monitoring packet, such as a monitoring packet comprising one or more of a sensing wire, for example a sterile sensing wire, and adhesive base, and a preferably sterile needle loaded into the delivery device.

### CROSS-REFERENCE TO RELATED APPLICATIONS

This document claims the benefit of priority to U.S. Provisional Application Ser. No. 63/489,491, filed Mar. 10, 2023, the contents of which are hereby incorporated by reference in their entirety.

### BACKGROUND

Accurate, long term analyte monitoring is of tremendous benefit to subjects such as subjects with diabetes, for which long term glucose measurement is of substantial value. Many current systems for delivery of monitoring devices are disposable after a single use, in that the monitoring device and its applicator are sterilely packed and discarded after a single use. These systems are both bulky and wasteful, as an applicator must be carried for each monitoring device, only to be discarded after a single use. Disposable, entirely single use systems add cost and effort to long term analyte efforts.

### SUMMARY

Disclosed herein are systems, methods and devices related to analyte monitoring devices, such as monitoring devices that effect continuous analyte monitoring. Systems, methods and devices herein often comprise a delivery device and a monitoring packet, such as a monitoring packet comprising one or more of a sensing wire, for example a sterile sensing wire, and adhesive base, and a preferably sterile needle loaded into the delivery device. Through practice of the methods or implementation of the delivery device, the needle is used to direct or to introduce the sensing wire at a site of a subject. The sensing wire is then dislodged from the needle, such that the sensing wire remains in situ, often stabilized by the adhesive base co-administered or previously or subsequently administered. The needle is then withdrawn back into the delivery device, and discarded. Resulting from the use of the systems, methods and devices herein, a monitoring device comprising at least one monitoring or sensing wire introduced into a subject is stably attached to a user, while a needle used to deliver a monitoring wire is removed from the user and discarded from the delivery device, such that the delivery device is recoverable for repeated use upon providing a second monitoring packet including a needle.

The monitoring packet is preferably sterile as it is applied at the time of administration. Monitoring packets comprise at least one sensing wire, in some cases comprising two or three separate sensing wires. Sensing wires variously serve as a sensing electrode, a working electrode, a reference electrode, a counter electrode or other electrode purpose.

The sensing wire is often coupled, such as electrically coupled, to a data collection monitoring moiety. In some cases the sensing wire is soldered to the data collection moiety. The data collection moiety is a component of the monitoring packet in some cases, while alternately some data monitoring packets are distinct from the monitoring packet.

The monitoring packet comprises an adhesive base, to which in some cases a cap is affixed, such as subsequent to adherence to the subject. The cap, unlike the base, is in many embodiments not sterile. Exemplary caps comprise PCBA. Affixing the cap variously comprises screwing the cap comprising a module onto the adhesive base using a screw thread on the upper surface of the adhesive base or on the cap. Often, the cap does not protrude beyond the upper surface of the adhesive base. Caps preferably do not comprise outward facing sharp edges. Caps often exhibit a height that is no more than, for example, 25% of its widest dimension, such as no more than 10%, 15%, 20%, 25%, 30%, 35%, 40%, or 50%. The effect of these configurations is create a smooth, low profile on the cap so as to prevent the cap and the monitoring device generally from catching onto clothing worn by a user over the monitoring device. Often, the cap comprises a rounded outward facing base so as to allow a layer of clothing to pass over the outward facing base without catching on the cap or the monitoring device. This facilitates long term continuous monitoring by the monitoring device in some cases.

The adhesive base comprises in some cases a computation module to process information generated by the sensing wire. The computation module may comprise a battery so as to, for example, power the processing of information.

Alternately, the computation module is in some cases a constituent of the cap, such that the cap comprises a computation module. Again, the computation module may comprise a battery so as to, for example, power the processing of information.

The cap in some cases comprises reusable electronics, consistent with the activities above and mentioned elsewhere herein.

Cap and the adhesive base are in some cases connected through connectors, such as 1, 2, 3, 4 or more than 4 connectors. In particular, some caps are connected to base via two batter connectors and two sensor connectors, so as to facilitate exchange of energy and information between base and cap.

Cap and base are attached through various approaches consistent with the disclosure herein. Caps are often connected through a thread-screw mechanism, such that the cap is screwed onto the base. Alternately, or in combination, a cap may be attached to base using a clip or magnet so as to facilitate both attachment to the base and connection of batter and signal communication.

Electrical connections, such as between cap and base, may be effected through a number of approaches consistent with the disclosure herein. Connection may be effected or maintained through pins so as to physically hold electrical connections in place while in some cases providing an avenue for current flow. Alternately or in combination, a magnet may be used to maintain contact among conductive materials such as gold or conductive carbon, or other suitable materials. Similarly, contact may be maintained through pressure contact so as to maintain an electrical connection.

One feature of some computation modules is to assess analyte monitoring accuracy. Another feature is to send a signal indicating a decline of analyte monitoring accuracy, such as a decline of analyte monitoring accuracy below a threshold.

An adhesive base remains on a subject for, for example, at least 4, 5, 6, 7, 8, 9, 10, 11, 12, 13, 14, or more than 14 days.

Sensing wires of the disclosure herein may in some cases comprise a sensing layer and a membrane layer.

Information generated by the sensing wire is transmitted in some cases to an external output device.

The adhesive base is often delivered by a delivery device concurrently to the subject as the needle delivers the sensing wire. This is performed in a process that may be repeated iteratively by a delivery device, without intervening sterilization of the delivery device. That is, a delivery device may be used to deliver two separate monitoring devices at two distinct time points, often without intervening sterilization of the delivery device.

The delivery device, in contrast, need not be sterile in various embodiments. Rather, it is often not sterile and is used for iterative administration of multiple monitoring devices in series to one or more subjects, such as replacement of exhausted or degraded monitoring devices over time to a subject. So long as a monitoring device packet is sterile, such as one comprising an adhesive layer, a sensor wire and a needle, the monitoring device can be sterilely delivered to a subject using a nonsterile, repeatably usable delivery device.

A feature of the delivery device is that it delivers an adhesive layer, a sensor wire and a needle to a subject, releases the wire from the needle, withdraws the needle from the subject and then discharges the needle from the delivery device, such that the delivery device is recovered unchanged relative to its starting configuration at the end of each delivery cycle. This facilitates the iterative re-usability of the delivery device. This reusability is further facilitated by the delivery device not needing to be sterile, such that re-sterilization of the delivery device is not required to deliver a sterile monitoring packet to a subject.

Devices herein in some cases benefit from waterproofing, to protect against fowling by human subject perspiration, to protect against external water sources as may contact devices herein pursuant to bathing, aquatic activity or adverse weather. Waterproofing variously comprises employing one or more of magnetic seals to maintain structural integrity of seals, alone or in combination with clips or screws. Such binders may further be supported by waterproof gaskets to seal junctions, as well as or in combination with the use of hydrophobic materials, particularly but not necessarily exclusively on the exterior of the device.

Also disclosed herein are analyte monitoring devices, such as continuous analyte monitoring devices, for application to a subject. Monitoring devices consistent with the disclosure herein variously comprise one or more of adhesive layer, a base, a battery, a contact and a sensor wire, an o-ring and a cap. The cap or the device as a whole is configured in some cases so as facilitate clothing being worn over the monitoring device.

Accordingly, monitoring devices variously comprise a cap having a z-profile of no greater than 0.25× that of the cap diameter. The cap variously does not protrude beyond the perimeter of the adhesive base, and in some cases comprises a rounded outward facing base.

The base in some cases comprises a computation module to process information generated by the sensing wire. The base is often configured to communicate with an external data presenting device.

The adhesive layer is configured to be stable upon extended application to a subject, such that it is stable to at least 2, 3, 4, 5, 6, 7, 8, 9, 10, 11, 12, 13, 14 or more than 14 days of sustained contact with a user.

The sensor wire is in some cases physically connected to a data collection moiety, such as being soldered to the data collection moiety or clamped on to a data collection moiety.

Some devices comprise a second sensor wire, such as in configurations where a first sensing wire is a working electrode and the second sending wire is a reference electrode, a counter electrode, or a reference and counter electrode. Some devices comprise a third electrode, such as a third electrode that comprises a counter.

In some cases the sensing wire comprises a sensing layer and a membrane layer.

Also disclosed herein are continuous analyte monitoring device delivery systems, such as those comprising one or more of at least one sterile delivery packet comprising an adhesive layer, at least one sensor wire, and a needle; a cap configured to attach to the adhesive layer; and an insertor assembly configured to receive the sterile packet, apply the sterile delivery packet adhesive layer to a surface, introduce the needle and the sensor wire through the surface, selectively withdraw the needle from the surface such that the sensor wire remains underneath the surface, withdraw the needle from the sterile packet, and disengage from the sterile packet.

Often, the delivery system or the insertor assembly system and the cap are nonsterile. Or are not required to be sterile.

The sterile delivery packet optionally comprises a data reception moiety, and in some cases a data transmission moiety. The data collection moiety is in some cases physically attached to the sensor wire, such as via soldering or is clamped to the sensor wire. In some cases the data collection moiety is clamped to the sensor wire subsequent to delivery of the sensor wire underneath the surface.

Some embodiments of the sterile delivery packet comprise a second needle and a second sensor wire. The second needle is in some cases separated from the needle by at least 1 mm, such as 1, 2, 3, 4, 5, 6, 7, 8, 9, 10 15, 20 30, 40, 50 or more than 50 mm. The sensing wire is in some configurations a working electrode and the second sensing wire is a reference electrode.

Some configurations further comprise a third wire, such as a third wire comprising a counter electrode.

Some sterile delivery packets comprise a cap docking port, such as a screw thread.

The needle of a delivery packet such as a sterile delivery packet in some cases comprises a longitudinal groove, such as a longitudinal groove configured to house the sensor wire. The sensor wire is in some cases dislodged from the longitudinal groove prior to or pursuant to removal of the needle from the subject, such that the sensor wire remains within the subject pursuant to removal of the needle from the subject and discarding the needle from the delivery device so as to recover a delivery device configured for delivery of a second delivery packet.

The insertor assembly is often configured to selectively withdraw the needle from the surface such that the sensor wire remains underneath the surface, often because the insertor assembly is configured to dislodge the sensor wire from the needle. The insertor assembly, to selectively withdraw the needle from the surface such that the sensor wire remains underneath the surface, is configured to dislodge the sensor wire from the needle longitudinal groove.

Furthermore, the insertor assembly is often configured to discard the needle from the insertor assembly, so as to return the delivery device to a configuration for delivery of a second delivery packet.

The cap configured to attach to the adhesive base in some cases comprises one or more of a battery, a data reception moiety, a data transmission moiety, a PCBA and an o-ring.

Exemplary cap embodiments are configured to have a low profile, suitable for deployment under clothing, such that for example in some cases the cap does not project beyond the perimeter of the adhesive layer, or wherein the cap has a height of no more than 25% of the width of the adhesive layer.

The cap attaches to the cap docking port, for example, reversibly attaches to the docking port such as through a complementary screw thread.

Often, attaching the cap to the adhesive layer assembles the continuous analyte monitoring system, or attaching the cap to the docking port assembles the continuous analyte monitoring system, or attaching the cap to the adhesive layer assembles the continuous analyte monitoring system.

The cap is configured for repeated use, such as repeated use without sterilization.

Systems herein often comprise a plurality of sterile packets.

Similarly, the insertor assembly is often configured for repeated use, such as repeated use without sterilization.

Exemplary systems herein comprise a data transmission receiving unit, such as a data transmission receiving unit that displays the data. The data is in exemplary embodiments not averaged prior to being displayed. This allows streamlined systems not requiring computational modules. Alternately, computational modules are included in some embodiments.

Also disclosed herein are dataset such as those generated by the systems disclosed herein, or datasets arising from continuous analyte monitoring, as well as methods of generating such datasets, for example using the systems herein, and methods of using the datasets, such as methods comprising detecting a measurement above, below or exceeding a threshold, or otherwise indicating intervention, and initiating a reagent administration or signaling such a measurement to a user.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

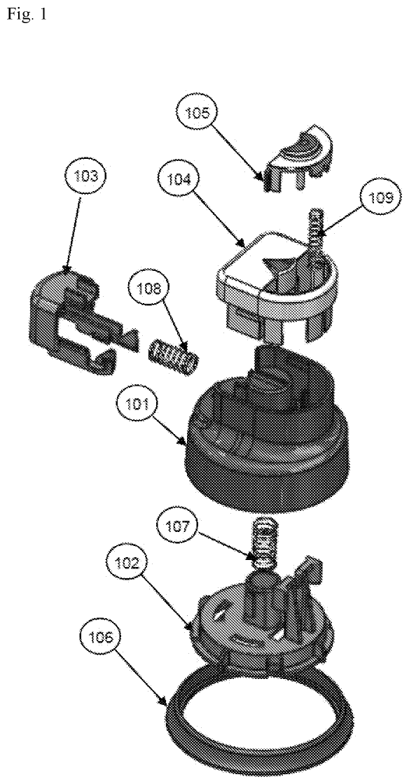

At FIG. 1, one sees an applicator assembly.

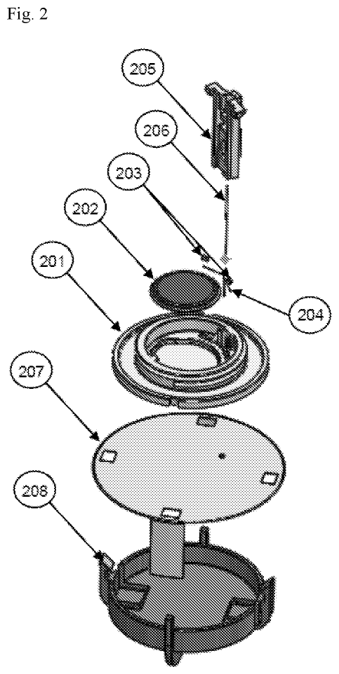

At FIG. 2 one sees a sensor assembly.

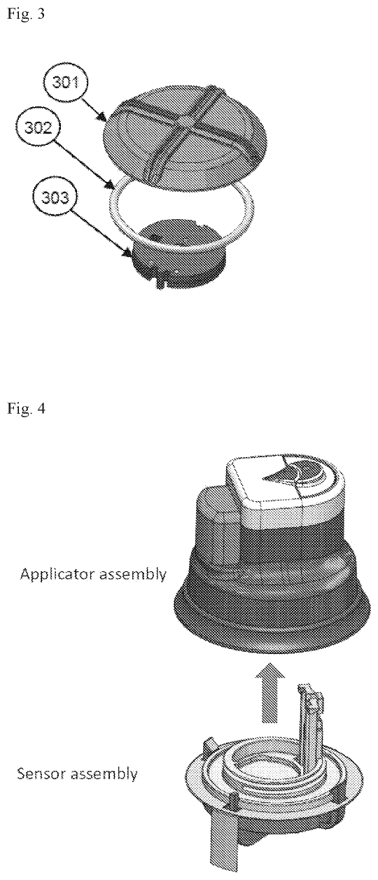

At FIG. 3 one sees an electronics assembly.

At FIG. 4 one sees a sensor assembly.

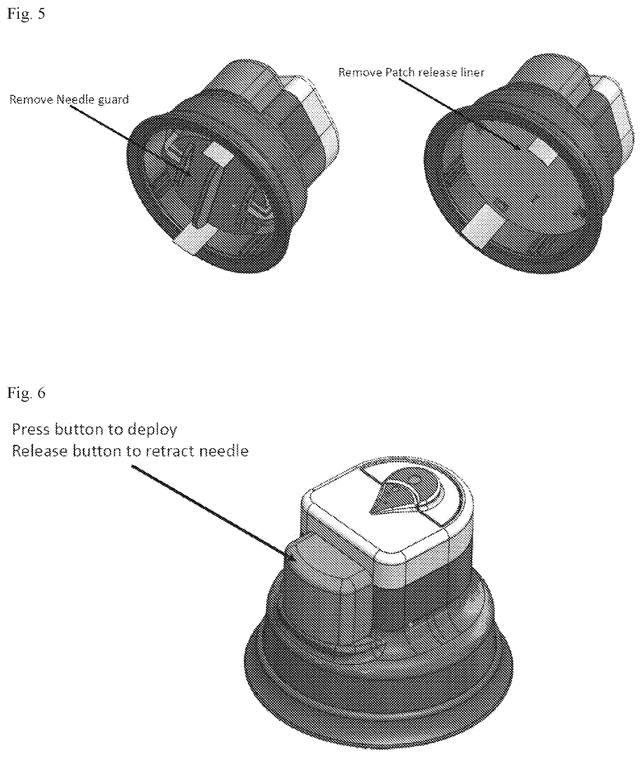

At FIG. 5 one sees an applicator assembly into which a sensor assembly has been loaded.

At FIG. 6 one sees an applicator assembly.

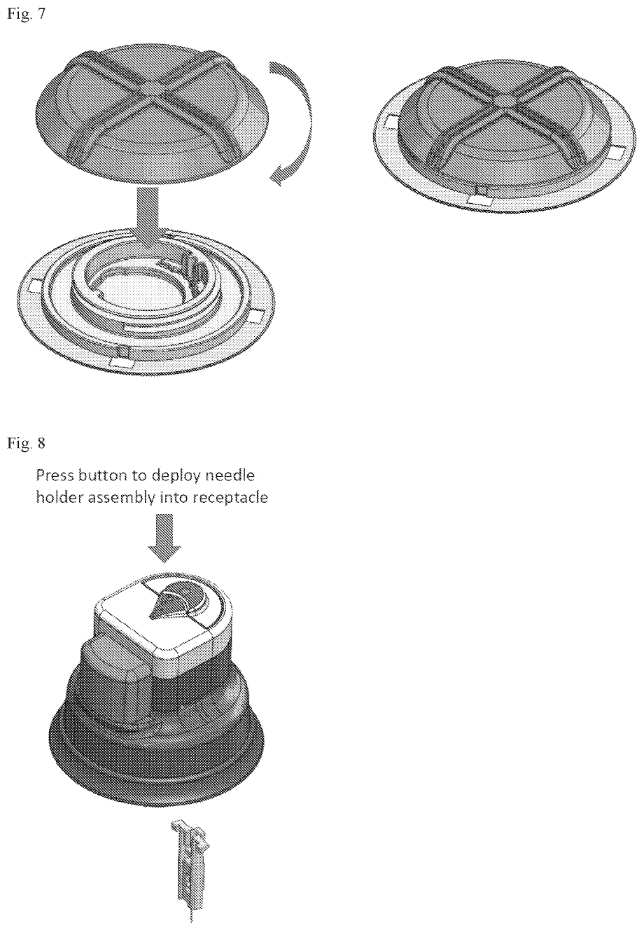

At FIG. 7 one sees a cap configured above a sensor assembly base (left) and screwed onto the sensor assembly via interlocking grooves, (right).

At FIG. 8 one sees an applicator assembly with the loaded sensor assembly needle holder particularly presented.

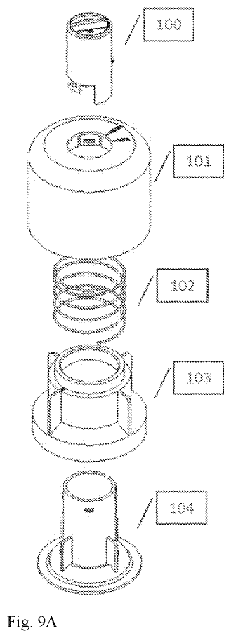

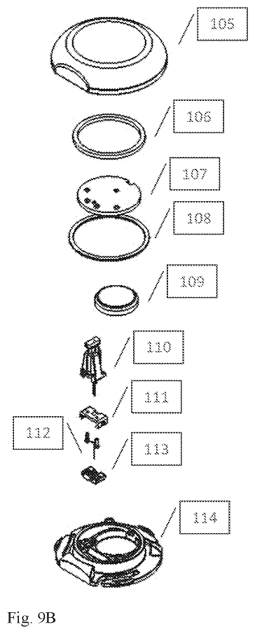

At FIG. 9A one sees a portion of an alternate applicator assembly.

At FIG. 9B, one sees a continued portion of an alternate applicator assembly.

### DETAILED DESCRIPTION

Disclosed herein are systems, devices and methods related to the application of a monitoring system such as a continuous monitoring system for long term use on a subject. Through use of the systems and devices and practice of the methods herein, an applicator is iteratively used to apply a series of monitoring system embodiments to a subject. The monitoring systems are sterile when applied to the subject, but the applicator need not be sterile, and need not be sterilized between repeated uses to apply monitoring systems to the subject.

The monitoring systems applied through the disclosure herein are often configured to lay close to the surface of the boy of a subject, so as to facilitate long term use without being bumped, jarred, disrupted or caught on subject clothing that may be worn over the monitoring systems. Accordingly, the monitoring systems often have one or more of a low profile, smoothed edges, or absence of right or acute angles configured radially from the center of the monitoring system caps. Monitoring systems often exhibit a height of no greater than 25% of their width or length, so as to exhibit a low profile that is less likely to be disrupted by contact or by clothing worn over the monitoring systems.

Accordingly, the systems, devices and methods herein are conducive to the long-term monitoring, such as continuous monitoring, of an analyte in a subject. Long term use is facilitated by the low profile of the individual monitoring systems, while long term use of systems comprising multiple monitoring systems is facilitated by the reusability of the applicators, which allow a subject to carry substantially more, smaller sterilely, individually packed monitoring systems because the monitoring systems do not need to be co-packaged with single use sterile applicators. Accordingly, the systems are more easily transported or carried for long term use or use remote from a storage site.

Systems herein also facilitate long-term use in some cases by being waterproof, so as to be compatible with exercise, or contact to external water sources as may occur through bathing, aqueous activity or adverse weather conditions. Waterproofing is effected variously through magnetic seals, water repellant or resistant gaskets, and through the use of hydrophobic materials.

The applicator of the systems, methods and devices herein is preferably re-usable, such that it need not be disposed of after a single use. Reuse does not require sterilization in some cases. Accordingly, systems and methods using the applicator disclosed herein require substantially less space to store or carry, and waste substantially less material, than do systems comprising single use applicator-monitoring system packets.

The applicator of the systems, methods and devices herein delivers a wire such as a sensing wire into a subject so as to facilitate analyte monitoring, such as continuous analyte monitoring. Often, the wire is sterile so as to facilitate long-term deposition into the subject. The applicator, in contrast, need not be sterile and is often not sterile. Rather, the applicator is preserved without intermittent sterilization so that it may be re-used for the iterative application of multiple monitoring packets to a subject.

Exemplary applicators are shown in FIG. 1. However, variants accomplishing comparable or similar outcomes are similarly contemplated herein.

A common feature of many applicators disclosed herein is their ability to insert a wire-loaded needle into a subject, dislodge the wire from the needle, dislodge or remove the wire from the needle, remove the needle from the subject and discard the needle from the applicator, such that the applicator is recovered in its initial state at the completion of an application event, thereby facilitating the re-use of the applicator.

Various applicators herein comprise spring-loaded needle insertion and removal moieties, in some cases powered by depressing of a button by a user such as the subject, as seen in FIG. 6.

Accordingly, disclosed herein are systems, devices and methods of delivering a monitoring device to a subject comprising using a re-usable applicator device to deposit a monitoring packet, such as a sterile monitoring packet, to a subject.

Applicators contemplated herein are configured to interface, interact with or be ‘loaded up by’ adding a monitoring packet to the applicator prior to use, as shown for example in FIG. 6. The monitoring packet is often sterilely wrapped, and unwrapped only prior to application to a subject.

The monitoring packet, embodiments of which are seen for example in FIG. 2 and FIG. 6, interfaces with the applicator to secure and deliver the monitoring device to the surface of the subject. The monitoring packet, unlike the applicator, is stored sterile and is single use. Monitoring packets generally comprise an adhesive surface for affixing the monitoring device to the subject, a base onto which measuring or computation moieties may be attached, and one of more wires, such as a sensing wire or working electrode, second wire or reference electrode, and third wire or counter electrode. In various embodiments the wires are separated by a minimum distance from one another, such as at least 0.5 mm, 1 mm, 2 mm, 3 mm, 4 mm, 5 mm or greater than 5 mm.

Most of the contents of the monitoring packet remain affixed to the subject. However, the needle or needles (generally one per wire to be introduced into the subject) are sterilely packaged, such as in the monitoring packet, but are discarded from the monitoring device upon introduction of the sensing or other wires into the subject as disclose below.

A needle of the one or more needles of the systems, methods and devices herein are configured for delivery of a wire such as a sensing wire to a subject. A needle of the system herein is preferably sterile and bundled with a monitoring packet. However, unlike much of the monitoring packet, the needle is separable, such that while the majority of the monitoring packet is adhered to the subject and remains stably attached until the monitoring device degrades, the needle is removed subsequent to deposition of a sensing or other wire in the subject.

Pursuant to deposition of a sensing wire, the needle is often configured to accommodate a sensing wire, and then to release from the sensing wire once the sensing wire is deposited in the subject. A number of needle configurations are consistent with this function of needles in the systems methods and devices herein. In some such configurations a needle comprises a groove running parallel along part or all of the length of the needle. The groove is configured to accommodate partially or fully a sensing wire threaded into the groove, for example such that the sensing wire and needle are parallel along the longest axis of the needle. In some cases the groove is excised from the exterior of the needle. Alternately, some needles effectively comprise a hollow longitudinal core and are further sliced longitudinally, so as to present a semicircular or other “c” configuration or incomplete circle cross-section.

The wire, such as a sensing wire, second wire or third wire such as a counting wire is threaded in the groove in the monitoring packet. Upon insertion of the wire into the subject, the wire is dislodged from the groove, such that when the needle is removed from the subject, the wire remains deposited within the subject and is available for sensing or other activity.

Alternate configurations through which the wire is deposited are also consistent with the disclosure herein. For example, in alternate embodiments, the wire is held to the needle through magnetic attraction or through a weakly bound chemical fixative that is readily degraded upon contacting to the interior of a subject.

Alternate wire deposition approaches are also contemplated, such as approaches wherein a wire comprises a rigid frame or coating, optionally degradable, or wherein the wires comprise a structural rigidity that facilitates deposition in the absence of or in combination with system disclosed herein or otherwise known to one of skill in the art.

A common feature of many of such needle and wire embodiments is that the wire remains in the subject, while the needle is removed and discarded by an applicator assembly, such as a reusable applicator assembly.

A sensing wire measures an analyte level, often continuously or incrementally measuring the analyte level, in some cases over prolonged durations. Exemplary analytes are glucose, among others as discussed below. The sensor wire measurements are processed on the monitoring device and transmitted to an external device, or transmitted to an external device prior to any processing. Exemplary external devices include wireless devices such as portable phones, as well as laptop or desktop computational devices. An external device may provide an output indicating the measurements obtained from the monitoring device such as a continuous monitoring device. Often, in addition to the output indicating measurements, the external device, or the monitoring device, may indicate whether threshold levels have been surpassed or reached, which may warrant an intervention such as glucose uptake or insulin release so as to maintain a physiologically desirable glucose level.

In some cases a monitoring device provides output to a reservoir such as an insulin or glucose reservoir on a subject, such that contents of such as reservoir, such as insulin or glucose or other reagent to address or respond to a measurement, may be directly provided to the subject, via injection, for example, so as to moderate analyte levels related to such measurement, such as glucose levels. This moderation may be automated or may be limited to notification of the subject so that the subject may effect manual or supervised moderation of the analyte levels such as glucose levels. A number of reservoirs such as insulin or glucose reservoirs are consistent with the disclosure herein.

In some cases a monitoring device provides output to a third party monitor, such as a monitor that may indicate a target analyte is present above, at or below a threshold. The analyte may in some cases be an anesthetic, such as may be administered to a patient pursuant to a surgical intervention. Alternately, the analyte may be a drug the abuse of which is to be monitored.

The cap, unlike the sensor assembly delivered in the monitoring packet, need not be sterile. The cap, such as the exemplary cap depicted in FIG. 3, is attached to the monitoring packet base affixed to the subject, such as via an adhesive. The cap is in some cases screwed on to the monitoring packet base.

As mentioned above, the cap is in some cases configured to have a low, smooth profile to facilitate worn clothing to pass over the cap without catching, jarring or otherwise disrupt the positioning of the monitoring device on the subject.

The cap often harbors an energy source such as a battery. Alternately, as shown in FIG. 2, the battery is delivered as part of the monitoring packet, bundled into the sensor assembly. In these cases the cap secures the battery in the sensor assembly.

The adhesive base comprises in some cases a computation module to process information generated by the sensing wire. The computation module may comprise a battery so as to, for example, power the processing of information.

Alternately, the computation module is in some cases a constituent of the cap, such that the cap comprises a computation module. Again, the computation module may comprise a battery so as to, for example, power the processing of information.

The cap in some cases comprises reusable electronics, consistent with the activities above and mentioned elsewhere herein.

Cap and the adhesive base are in some cases connected through connectors, such as 1, 2, 3, 4 or more than 4 connectors. In particular, some caps are connected to base via two batter connectors and two sensor connectors, so as to facilitate exchange of energy and information between base and cap.

Cap and base are attached through various approaches consistent with the disclosure herein. Caps are often connected through a thread-screw mechanism, such that the cap is screwed onto the base. Alternately, or in combination, a cap may be attached to base using a clip or magnet so as to facilitate both attachment to the base and connection of batter and signal communication.

Electrical connections, such as between cap and base, may be effected through a number of approaches consistent with the disclosure herein. Connection may be effected or maintained through pins so as to physically hold electrical connections in place while in some cases providing an avenue for current flow. Alternately or in combination, a magnet may be used to maintain contact among conductive materials such as gold or conductive carbon, or other suitable materials. Similarly, contact may be maintained through pressure contact so as to maintain an electrical connection.

One feature of some computation modules is to assess analyte monitoring accuracy. Another feature is to send a signal indicating a decline of analyte monitoring accuracy, such as a decline of analyte monitoring accuracy below a threshold.

An adhesive base remains on a subject for, for example, at least 4, 5, 6, 7, 8, 9, 10, 11, 12, 13, 14, or more than 14 days.

Sensing wires of the disclosure herein may in some cases comprise a sensing layer and a membrane layer.

Information generated by the sensing wire is transmitted in some cases to an external output device.

The adhesive base is often delivered by a delivery device concurrently to the subject as the needle delivers the sensing wire. This is performed in a process that may be repeated iteratively by a delivery device, without intervening sterilization of the delivery device. That is, a delivery device may be used to deliver two separate monitoring devices at two distinct time points, often without intervening sterilization of the delivery device.

The delivery device, in contrast, need not be sterile in various embodiments. Rather, it is often not sterile and is used for iterative administration of multiple monitoring devices in series to one or more subjects, such as replacement of exhausted or degraded monitoring devices over time to a subject. So long as a monitoring device packet is sterile, such as one comprising an adhesive layer, a sensor wire and a needle, the monitoring device can be sterilely delivered to a subject using a nonsterile, repeatably usable delivery device.

A feature of the delivery device is that it delivers an adhesive layer, a sensor wire and a needle to a subject, releases the wire from the needle, withdraws the needle from the subject and then discharges the needle from the delivery device, such that the delivery device is recovered unchanged relative to its starting configuration at the end of each delivery cycle. This facilitates the iterative re-usability of the delivery device. This reusability is further facilitated by the delivery device not needing to be sterile, such that re-sterilization of the delivery device is not required to deliver a sterile monitoring packet to a subject.

Devices herein in some cases benefit from waterproofing, to protect against fowling by human subject perspiration, to protect against external water sources as may contact devices herein pursuant to bathing, aquatic activity or adverse weather. Waterproofing variously comprises employing one or more of magnetic seals to maintain structural integrity of seals, alone or in combination with clips or screws. Such binders may further be supported by waterproof gaskets to seal junctions, as well as or in combination with the use of hydrophobic materials, particularly but not necessarily exclusively on the exterior of the device.

The durability and reusability of the applicator in the disclosure herein has a number of implications for systems and methods in which it is incorporated.

In particular, systems may be packaged or sold comprising an unequal proportion of applicators and monitoring packets. That is systems may be packaged, sold or used having a ratio of monitoring packets to applicators of 2:1, 3:1, 4:1, 5:1, 6:1, 7:1, 8:1, 9:1, 10:1, 20:1 or greater. That is, some systems may comprise one or two applicators, but substantially more monitoring packets than applicators, such as 5, 6, 7, 8, 9, 10 or more than 10 monitoring packets. The monitoring packets are individually sterilely wrapped or stored, while the applicator may optionally be initially sterile, but need not be sterile to function in delivering successive monitoring packets to a subject.

Similarly, systems may be packaged or sold comprising an unequal proportion of caps and monitoring packets. That is systems may be packaged, sold or used having a ratio of monitoring packets to caps of 2:1, 3:1, 4:1, 5:1, 6:1, 7:1, 8:1, 9:1, 10:1, 20:1 or greater. That is, some systems may comprise one or two reusable caps, but substantially more monitoring packets than caps, such as 5, 6, 7, 8, 9, 10 or more than 10 monitoring packets. The monitoring packets are individually sterilely wrapped or stored, while the cap may optionally be initially sterile, but need not be sterile to function in capping or being screwed onto successive monitoring packets on a subject.

The systems, devices and methods consistent with this disclosure facilitate long term, efficient use of and replacement of monitoring devices on the surface of a subject. They enable methods comprising the iterative use of a nonsterile applicator to position a sterile sensor assembly onto which a monitoring device may be assembled. The deposition of this sensor assembly comprises inserting one or more wires, such as a sensor wire, into a subject. The deposition often comprises use of a needle, such as a grooved needle, so as to insert the wire into a subject, dislodge the wire from the needle, remove and then discard the wire from the applicator, such that the applicator is returned to its initial state and is available for repeated use.

Some systems further comprise an analyte level moderator component, such as an analyte reservoir or an analyte metabolizing reagent reservoir. This moderator component is in some cases in wireless or direct communication with the monitoring component, such that the moderator component may automatically release an analyte such as glucose or an analyte moderator such as insulin directly, in an automated fashion, in response to the analyte levels measured by the measuring device. Alternately, some analyte level moderator components are configured for manual rather than automatic operation, or a combination of manual and automatic operation, such that a subject may receive analyte monitoring level outputs but may nonetheless preserve total or partial control over release or application of analyte moderating components such as glucose or insulin.

A number of analytes may be detected using the systems, devices and methods herein. An exemplary analyte target is glucose, such as is measured pursuant to the modulation of fluctuation in glucose levels as may occur in individuals diagnosed with or exhibiting traits of diabetes.

Other analytes are also consistent with the disclosure herein. Alcohol levels or controlled substance levels may be measured, such as pursuant to an effort to document use or to facilitate cessation of use. Similarly, levels of a drug such as an anesthetic or an antibiotic may be measured pursuant to a surgical procedure, for example so as to assess the effective dose to which a subject is exposed throughout a surgical procedure, or to detect a level of drug that may be detrimental to the recipient. Hormones such as those mediating fertility may also be measured, such as continuously measured using the technology herein. Similarly, behavioral hormones such as adrenalin, testosterone, or serotonin levels may be measured in an effort to assess behavior or mental health. Additional target analytes are also consistent with the disclosure herein.

Turning to the Figures, one sees the following.

At FIG. 1, one sees an applicator assembly. The applicator assembly does not need to be sterile to effect sterile application of a continuous monitoring device, and the applicator assembly is configured for iterative re-use. 101 depicts the applicator assembly housing. 102 represents an applicator slide, having a slide spring 107, that interfaces with a cushion 106. The button trigger 103 interacts with a second spring 108. The applicator cap housing 104 houses the needle dispenser spring 109. Resting above the applicator cap housing is the needle disposal button 105.

At FIG. 2 one sees a sensor assembly. The sensor assembly comprises a needle protector 208 covering a patch adhesive 207. The adhesive is exposed prior to application to a subject. The adhesive is below a base 201, that is grooved to receive a screw-cap 301-303, and harbors a battery 202. Also deployed in the base 201 is a contact 203 and a sensor wire 204. In various embodiments a second sensor wire and in some cases a third sensor wire (not shown) are also configured about the base 201. The needle 206 is delivered through a needle holder 205, such that the needle delivers the sensor wire 204 to a subject. The needle often comprises a groove, such that the sensor wire is threaded into the groove for delivery and released from the groove upon removal of the needle so as to allow retention of the wire upon removal of the needle from the subject.

At FIG. 3 one sees an electronics assembly, comprising a cap 301, an o-ring 302 and a PCBA 303. Notably, the cap has a low profile and trimmed edges such that the cap is suitable for deployment onto a sensor assembly under the clothes of a subject without catching the clothing of the subject.

Table 1 presents the components of FIG. 1, FIG. 2, and FIG. 3.

TABLE 1

| Item # | Description | Qty |
|---|---|---|
| 100 | APPLICATOR ASSEMBLY | 1 |
| 101 | HOUSING | 1 |
| 102 | SLIDE | 1 |
| 103 | BUTTON, TRIGGER | 1 |
| 104 | CAP, HOUSING | 1 |
| 105 | BUTTON, NEEDLE DISPOSAL | 1 |
| 106 | CUSHION | 1 |
| 107 | SPRING, SLIDE | 1 |
| 108 | SPRING, BUTTON, TRIGGER | 1 |
| 109 | SPRING, BUTTON, NEEDLE DISPOSAL | 1 |
| 200 | SENSOR ASSEMBLY | 1 |
| 201 | BASE | 1 |
| 202 | BATTERY | 1 |
| 203 | CONTACT | 2 |
| 204 | SENSOR WIRE | 2 |
| 205 | HOLDER, NEEDLE | 1 |
| 206 | NEEDLE | 1 |
| 207 | PATCH, ADHESIVE | 1 |
| 208 | PROTECTOR, NEEDLE | 1 |
| 300 | ELECTRONICS ASSEMBLY | 1 |
| 301 | CAP | 1 |
| 302 | O'RING | 1 |
| 303 | PCBA | 1 |

At FIG. 4 one sees a sensor assembly, comprising a needle protector, a patch adhesive, a base and a needle holder, and an applicator assembly, comprising a base, a housing, a spring button, an applicator cap housing that houses the needle dispenser spring, and resting above the applicator cap housing is the needle disposal button. The sensor assembly is single use and sterile, while the applicator assembly need not be sterile and is configured for repeated use with multiple sensor assemblies.

At FIG. 5 one sees an applicator assembly into which a sensor assembly has been loaded. At left, the sensor assembly is loaded as packaged. At right, the sensor assembly needle protector and a patch adhesive liner are removed so as to expose the patch adhesive, which is sterile.

At FIG. 6 one sees an applicator assembly, comprising a base, a housing, a spring button, an applicator cap housing that houses the needle dispenser spring, and resting above the applicator cap housing is the needle disposal button. The applicator assembly need not be sterile and is configured for repeated use with multiple sensor assemblies.

At FIG. 7 one sees a cap configured above a sensor assembly base (left) and screwed onto the sensor assembly via interlocking grooves, (right). The cap is configured to have a low profile that does not interfere with clothing that may be worn over the device. The sensor assembly is sterile in exemplary embodiments, while the cap does not need to be sterile to effect sterile application of the sensor assembly. Not shown are the o-ring, PCBA and battery.

At FIG. 8 one sees an applicator assembly with the loaded sensor assembly needle holder particularly presented. The needle holder is deployed into the sensor assembly receptacle by pressing the needle disposal button.

At FIG. 9A and FIG. 9B one sees an alternate applicator assembly. FIG. 9A item 100 represents a button, needle disposal, lock and trigger. Item 101 depicts a housing for the button, needle disposal, lock and trigger. Item 102 represents a spring, which is enclosed in item 103, a spring housing. Item 104 depicts a sensor assembly mount. Item 105 represents a cover of housing. Item 106 represents an o-ring, 107 represents a PCBA, 108 represents a second o-ring, and 109 represents a battery. Item 110 represents a needle and holder, 111 represents a top mount, 112 presents a sensor wire, 113 presents a bottom mount, and item 114 presents a base with an adhesive patch.

A listing of its components is seen in accompanying Table 2.

TABLE 2

| Item # | Description |
|---|---|
| 100 | BUTTON, NEEDLE DISPOSAL, LOCK, TRIGGER |
| 101 | HOUSING |
| 102 | SPRING |
| 103 | SPRING HOUSING |
| 104 | SENSOR ASSEMBLY MOUNT |
| 105 | COVER, HOUSING |
| 106 | ORING |
| 107 | PCBA |
| 108 | ORING |
| 109 | BATTERY |
| 110 | NEEDLE AND HOLDER |
| 111 | TOP MOUNT |
| 112 | SENSOR WIRE |
| 113 | BOTTOM MOUNT |
| 114 | BASE WITH ADHESIVE PATCH |

### EXAMPLES

Example 1. A continuous monitoring device is delivered through reusable applicator. A monitoring packet comprising a sterile sensing wire, an adhesive base, and a sterile needle is loaded into a delivery device. The delivery device is not sterile.

By pressing a button on the delivery device, the adhesive base of the monitoring packet is applied to the skin of a subject, and a sensing wire is introduced into the subject, directed by the needle at a site on the subject. The sensing wire is dislodged from the needle and the needle is removed from the subject, drawn through the applicator and discarded

The applicator is recovered as it was configured prior to use, so that it may be reused to administer a second monitoring packet.

Only the monitoring packet contacts the subject's skin at the point of skin penetration. Accordingly, only the monitoring packet benefits from being sterile. The applicator need not be sterile, and may be reused without sterilization.

The monitoring packet is stored individually in a set of monitoring packets, individually sterilely wrapped so that a subject may replace monitoring packets as they wear out. The monitoring packets may be replaced using a single, nonsterile applicator.

The subject need only carry a single reusable applicator to be able to apply a plurality of sterile monitoring packets in succession as they wear out. The subject need not carry multiple applicators, which are generally much bulkier than are the monitoring packets, and the subject need not discard the applicator after each application of a monitoring packet. Accordingly, the system is substantially more convenient for a subject to carry and use long-term, because only a single applicator is needed to be carried. Similarly, the system has a substantially reduced waste burden, as the applicator is not single-use and need not be discarded with the frequency with which monitoring packets are exhausted.

Example 2. An alternative single use applicator is used to deliver a monitoring packet. The monitoring packet and applicator are bundled in a single sterile packet, which is opened immediately prior to use. The monitoring packet is applied and the applicator is discarded.

The size of each single sterile packet is substantially larger and more cumbersome than are the sterile monitoring packets in Example 1, which limits the number of packets than can be conveniently carried by a subject at a time. Furthermore, the applicators are discarded after each use, creating substantially more waste than created by the use of the system of Example 1.

Example 3. A continuous monitoring device is applied to a subject. The monitoring device comprises an adhesive layer, a base, a battery, a contact and a sensor wire delivered bundled in a sterile monitoring packet, and a cap unit comprising a cap, an o-ring and a PCBA. The cap unit is screwed into the base to assemble the continuous monitoring device.

The cap has a low profile relative to the width of the monitoring device base, such that the ratio of the cap height to width is no more than 0.25. The cap lacks sharp or right angle edges on a radial axis from the center of the cap attachment point.

Consequently, the cap is relatively smooth at the point of attachment, and readily remains attached to the subject even when worn under normal fitting clothing without ‘catching’ on the clothing.

We claim:

1. A method of applying an analyte monitoring device to a subject, comprising loading a monitoring packet comprising a sterile sensing wire, an adhesive base, and a sterile needle into a delivery device, concurrently applying the adhesive base to the skin of a subject and introducing a sensing wire directed by the needle at a site on the subject, removing the needle from the subject into the delivery device, such that the sensing wire remains in situ stabilized by the adhesive base, and discarding the needle from the delivery device.

2. The method of claim 1, wherein the monitoring packet is sterile as it is applied to the subject.

3. The method of claim 1, wherein the monitoring packet comprises a second sterile sensing wire.

4. The method of claim 3, wherein the sensing wire is a working electrode and the second sending wire is a reference electrode.

5. The method of claim 4, comprising introducing a third wire.

6. The method of claim 5, wherein the third wire comprises a counter electrode.

7. The method of claim 1, wherein the delivery device is not sterile.

8. The method of claim 1, wherein the delivery device does not need to be sterile for delivery of the monitoring packet comprising a sterile sensing wire, an adhesive base, and a sterile needle to the subject.

9. The method of claim 1, wherein the sterile sensing wire is electrically coupled to a data collection moiety of the monitoring packet.

10. The method of claim 1, wherein the sterile sensing wire is soldered to a data collection moiety of the monitoring packet.

11. The method of claim 1, comprising affixing a cap to the adhesive base, thereby assembling the continuous analyte monitoring device.

12. The method of claim 11, wherein the cap is not sterile.

13. The method of claim 11, wherein the cap comprises a PCBA.

14. The method of claim 11, wherein the cap comprises a battery.

15. The method of claim 11, wherein the cap comprises reusable electronics.

16. The method of claim 11, wherein the cap comprises at least one battery connector.

17. The method of claim 11, wherein the cap comprises two battery connectors.

18. The method of claim 11, wherein the cap comprises at least one sensor connector.

19. The method of claim 11, wherein the cap comprises two sensor connectors.

20. The method of claim 11, wherein the cap effects electrical connection with the base via at least one pin.

21. The method of claim 11, wherein the cap effects electrical connection with the base via at least one magnet.

22. The method of claim 11, wherein the cap effects electrical connection with the base via at least one pressure contact site.

23. The method of claim 11, wherein the cap effects electrical connection with the base via at least one conductive material selected from the list consisting of gold and carbon.

24. The method of claim 11, wherein affixing the cap comprises screwing the cap onto the base.

25. The method of claim 11, wherein affixing the cap comprises clipping the cap to the base.

26. The method of claim 11, wherein affixing the cap comprises magnetically connecting the cap to the base.

27. The method of claim 11, wherein affixing the cap comprises effecting a water-tight connection between the cap and the base.

28. The method of claim 1, wherein concurrently applying an adhesive base and introducing a sensing wire directed by a removable needle as a site on the subject comprises delivering the adhesive base and the removable needle via a reusable insertor assembly.

29. The method of claim 28, wherein the reusable insertor assembly is employed to apply two separate continuous analyte monitoring devices to a subject at two distinct time points.

30. The method of claim 1, wherein affixing the computation module comprises screwing the computation module onto the adhesive base using a screw thread on the upper surface of the adhesive base.

31. The method of claim 1, wherein the cap does not protrude beyond the perimeter of the adhesive base.

32. The method of claim 1, wherein the cap does not comprise an outward facing sharp edge.

33. The method of claim 1, wherein the cap height is no more than 25% of its widest dimension.

34. The method of claim 1, wherein the cap comprises a rounded outward facing base so as to allow a layer of clothing to pass over the outward facing base without catching.

35. The method of claim 1, comprising transmitting information generated by the sensing wire to an external output device.

36. The method of claim 1, wherein the adhesive base comprises a computation module to process information generated by the sensing wire.

37. The method of claim 36, wherein the computation module comprises a battery.

38. The method of claim 1, wherein the cap comprises a computation module to process information generated by the sensing wire.

39. The method of claim 38, wherein the computation module comprises a battery.

40. The method of any one of claims 36 to 39, wherein the computation module assesses analyte monitoring accuracy.

41. The method of claim 40, wherein the computation module sends a signal indicating decline of analyte monitoring accuracy below a threshold.

42. The method of claim 1, wherein the adhesive base forms a water-tight connection to the subject.

43. The method of claim 1, wherein the adhesive base remains on the subject for at least 4 days.

44. The method of claim 1, wherein the adhesive base remains on the subject for at least 7 days.

45. The method of claim 1, wherein the adhesive base remains on the subject for at least 14 days.

46. The method of claim 1, wherein the sensing wire comprises a sensing layer and a membrane layer.

47. A continuous analyte monitoring device, comprising an adhesive layer, a base, a battery, a contact and a sensor wire, an o-ring and a cap, wherein the cap does not protrude beyond the perimeter of the adhesive base, wherein the cap does not comprise an outward facing sharp edge, and wherein the cap comprises a rounded outward facing base so as to allow a layer of clothing to pass over the outward facing base without catching.

48. The device of claim 47, wherein the cap z-profile is no greater than 0.25× of the cap diameter.

49. The device of claim 47, wherein the base comprises a computation module to process information generated by the sensing wire.

50. The device of claim 47, wherein the base is configured to communicate with an external data presenting device.

51. The device of claim 47, wherein the cap connects to the base via a clip.

52. The device of claim 47, wherein the cap connects to the base via a screw connection.

53. The device of claim 47, wherein the cap connects to the base via a magnet.

54. The device of claim 47, wherein the cap comprises reusable electronics.

55. The device of claim 47, wherein the cap connects to the base via two battery connectors.

56. The device of claim 47, wherein the cap connects to the base via two sensor connectors.

57. The device of claim 47, wherein the cap connects the base electrically via pins.

58. The device of claim 47, wherein the cap connects the base electrically via a conductive material.

59. The device of claim 58, wherein the conductive material comprises gold.

60. The device of claim 58, wherein the conductive material comprises carbon.

61. The device of claim 47, wherein the cap connects the base electrically via a pressure contact.

62. The device of claim 47, wherein the adhesive layer is stable to at least 4 days of sustained contact with a user.

63. The device of claim 47, wherein the adhesive layer is stable to at least 7 days of sustained contact with a user.

64. The device of claim 47, wherein the adhesive layer is stable to at least 14 days of sustained contact with a use.

65. The device of claim 47, wherein the adhesive layer is stable to contact to water.

66. The device of claim 47, wherein the sensor wire is physically connected to a data collection moiety.

67. The device of claim 66, wherein the sensor wire is soldered to the data collection moiety.

68. The device of claim 47, wherein the sensor wire is clamped on to a data collection moiety.

69. The device of claim 47, wherein the device comprises a second sensor wire.

70. The device of claim 69, wherein the sensing wire is a working electrode and the second sensing wire is a reference electrode.

71. The device of claim 70, comprising a third wire.

72. The device of claim 71, wherein the third wire comprises a counter electrode.

73. The device of claim 47, wherein the sensing wire comprises a sensing layer and a membrane layer.

74. The device of claim 47, wherein the device is watertight.

75. The device of claim 47, wherein the device comprises hydrophobic material.

76. The device of claim 47, wherein the device comprises a watertight seal to the subject.

77. The device of claim 47, wherein the device comprises a watertight seal between the base and the cap.

78. A continuous analyte monitoring device delivery system, comprising at least one sterile delivery packet comprising an adhesive layer, at least one sensor wire, and a needle;
a cap configured to attach to the adhesive layer; and
a insertor assembly configured to receive the sterile packet, apply the sterile delivery packet adhesive layer to a surface, introduce the needle and the sensor wire through the surface, selectively withdraw the needle from the surface such that the sensor wire remains underneath the surface, withdraw the needle from the sterile packet, and disengage from the sterile packet.

79. The system of claim 78, wherein the cap is nonsterile.

80. The system of claim 78, wherein the insertor assembly is nonsterile.

81. The system of claim 78, wherein the sterile delivery packet comprises a data reception moiety.

82. The system of claim 78, wherein the sterile delivery packet comprises a data transmission moiety.

83. The system of claim 81, wherein the data collection moiety is physically attached to the sensor wire.

84. The system of claim 83, wherein the data collection moiety is physically attached to the sensor wire via soldering.

85. The system of claim 81, wherein the data collection moiety is clamped to the sensor wire.

86. The system of claim 85, wherein the data collection moiety is clamped to the sensor wire subsequent to delivery of the sensor wire underneath the surface.

87. The system of claim 78, wherein the sterile delivery packet comprises a second needle and a second sensor wire.

88. The system of claim 87, wherein the second needle is separated from the needle by at least 1 mm

89. The system of claim 87, wherein the sensing wire is a working electrode and the second sensing wire is a reference and counter electrode.

90. The system of claim 89, comprising a third wire.

91. The system of claim 90, wherein the third wire comprises a counter electrode.

92. The system of claim 78, wherein the sterile delivery packet comprises a cap docking port.

93. The system of claim 92, wherein the cap docking port comprises a screw thread.

94. The system of claim 78, wherein the needle comprises a longitudinal groove.

95. The system of claim 94, wherein the longitudinal groove is configured to house the sensor wire.

96. The system of claim 78, wherein the cap comprises a battery.

97. The system of claim 78, wherein the cap comprises a data reception moiety.

98. The system of claim 78, wherein the cap comprises a data transmission moiety.

99. The system of claim 78, wherein the cap comprises a PCBA.

100. The system of claim 78, wherein the cap comprises an o-ring.

101. The system of claim 78, wherein the cap does not project beyond the perimeter of the adhesive layer.

102. The system of claim 78, wherein the cap has a height of no more than 25% of the width of the adhesive layer.

103. The system of claim 92, wherein the cap attaches to the cap docking port.

104. The system of claim 103, wherein the cap reversibly attaches to the docking port.

105. The system of claim 93, wherein the cap comprises a complementary screw thread.

106. The system of claim 78, wherein attaching the cap to the adhesive layer assembles the continuous analyte monitoring system.

107. The system of claim 103, wherein attaching the cap to the docking port assembles the continuous analyte monitoring system.

108. The system of claim 105, wherein attaching the cap to the adhesive layer assembles the continuous analyte monitoring system.

109. The system of claim 78, wherein the insertor assembly, to selectively withdraw the needle from the surface such that the sensor wire remains underneath the surface, is configured to dislodge the sensor wire from the needle.

110. The system of claim 95, wherein the insertor assembly, to selectively withdraw the needle from the surface such that the sensor wire remains underneath the surface, is configured to dislodge the sensor wire from the needle longitudinal groove.

111. The system of claim 110, wherein the insertor assembly is configured to discard the needle from the insertor assembly.

112. The system of claim 78, comprising a plurality of sterile packets.

113. The system of claim 78, wherein the cap is configured for repeated use.

114. The system of claim 113, wherein the cap is configured for repeated use without sterilization.

115. The system of claim 78, wherein the insertor assembly is configured for repeated use.

116. The system of claim 115, wherein the insertor assembly is configured for repeated use without sterilization.

117. The system of claim 82, comprising a data transmission receiving unit.

118. The system of claim 117, wherein the data transmission receiving unit displays the data.

119. The system of claim 118, wherein the data is not averaged prior to being displayed.

120. The system of claim 98, comprising a data transmission receiving unit.

121. The system of claim 120, wherein the data transmission receiving unit displays the data.

122. The system of claim 121, wherein the data is not averaged prior to being displayed.
